SEQUENCE LISTING (2 sequences)

SEQ ID NO: 1 - DNA, 21 bp, Artificial Sequence
  Synthetic Primer
tggagagcaccaagacagaca

SEQ ID NO: 2 - DNA, 23 bp, Artificial Sequence
  Synthetic Primer
gtcgacaatgatgacatccttca

(19) United States
(12) Patent Application Publication
(10) Pub. No.: US 2015/0299954 A1
(43) Pub. Date: Oct. 22, 2015

(54) Nano-sized Bagasse Fiber

(71) Applicants: Kun Lian, Baton Rouge, LA (US); William T. Cefalu, Baton Rouge, LA (US); Zhong Q. Wang, Baton Rouge, LA (US)

(72) Inventors: Kun Lian, Baton Rouge, LA (US); William T. Cefalu, Baton Rouge, LA (US); Zhong Q. Wang, Baton Rouge, LA (US)

(73) Assignee: Board of Supervisors of Louisiana State University and Agricultural and Mechanical College, Baton Rouge, LA (US)

(21) Appl. No.: 14/754,881

(22) Filed: Jun. 30, 2015

Related U.S. Application Data
(62) Division of application No. 12/037,436, filed on Feb. 26, 2008, now abandoned.
(60) Provisional application No. 60/891,630, filed on Feb. 26, 2007.

(51) Int. Cl. D21H 11/12; A61K 8/97; A23L 1/308; A61K 8/02; A61Q 1/02; D21B 1/14; A61K 31/717

(52) U.S. Cl. CPC D21H 11/12; D21B 1/14; A61K 8/97; A61K 31/717; A61K 8/027; A61Q 1/02; A23L 1/3081; A61K 2800/413; A23V 2002/00

(57) ABSTRACT

A new composition of nanosized bagasse fibers has been made by a method which reduces the sugarcane bagasse fibers to nano-sized particles while retaining the natural components of the bagasse. The resulting bagasse particles were shown to be effective as a nutritional supplement in a mouse model to aid in glucose control and body weight. Using the bagasse nanofibers, the addition of 5 to 10% fiber did not change the color or texture of food products. Moreover, the bagasse powder has a natural color and absorbs color evenly so that it could be used as a natural foundation material for cosmetic products.

This is a divisional of co-pending application Ser. No. 12/037,436, filed Feb. 26, 2008, now abandoned; which claimed the benefit of provisional U.S. application Ser. No. 60/891,630, filed Feb. 26, 2007 under 35 U.S.C. §119(e); the complete disclosures of both of which are hereby incorporated by reference.

This technology pertains to nano-sized, unoxidized bagasse fibers for use as nutritional supplements and produced by pulverizing at cryogenic temperatures to preserve the natural components of bagasse.

### Sugarcane Bagasse Fibers

Sugarcane bagasse is produced as a by-product of the manufacture of raw sugar from sugarcane. Bagasse is a term used to designate the mostly woody fibrous residue after the juice has been extracted. Because of the large quantity of bagasse produced, much research is directed to finding new uses for this discarded material. Sugarcane bagasse fiber contains 46 wt % cellulose, 24.5 wt % hemicellulose, 19.95 wt % lignin, 3.45 wt % fats and waxes, 2.4 wt % ash, 2.0 wt % silicon, and 1.70 wt % other substances. Policosanol, a constituent of sugarcane waxes, has been found to have beneficial effects in both human and animal models. Policosanol has been reported to lower LDL and increase HDL in the plasma, and increase platelet aggregation. (See, e.g., G. Castano et al., Effects of policosanol and pravastatin on lipid profile, platelet aggregation and endothelemia in older hypercholesterolemic patients, Int. J. Clin. Pharmacol. Res., vol. 19, pp. 105-116 (1999)). Besides providing policosanol, sugarcane bagasse also is a unique source of organic fiber for human dietary supplement.

### Dietary Fiber

Numerous studies have examined the effects of macronutrients, that is, dietary fat, protein, and carbohydrates, on energy intake; but studies assessing the role of dietary fiber on this process are more limited [1]. Fiber is not considered an essential nutrient, but may play a role in modulation of energy intake and, in this regard, has been suggested to lower risk for developing obesity [2]. Dietary fibers, that is, the indigestible portion of plant foods, can be broadly classified as being either “soluble” or “insoluble” and “fermentable” or “nonfermentable.” Chemically, dietary fiber consists of non-starch polysaccharides and several plant components such as cellulose, lignin, waxes, chitins, pectins, β-glucans, inulin, and oligosaccharides. These fiber components have unique chemical structures and characteristic physical properties, for example, bulk/volume, viscosity, water-holding capacity, adsorption/binding, or fermentability, which determine their subsequent physiologic behavior.

The American Dietetic Association recommends a minimum of 20 to 35 g/d dietary fiber for a healthy adult [3], whereas the average American diet barely contains half this amount, for example, 10 to 15 g daily [4]. A significant relationship between lower intake of fiber and obesity has been suggested by epidemiologic and cross-sectional studies [5-7]. As such, increased intake of dietary fiber may offer additional health benefits to obese and diabetic patients. For example, dietary fiber supplementation was shown to significantly improve carbohydrate metabolism and insulin sensitivity in overweight and obese women [8]. In addition, a high intake of dietary fiber, particularly of the soluble type, improved glycemic control, decreased hyperinsulinemia, and lowered plasma lipid concentrations in patients with type 2 diabetes mellitus [9]. These benefits of increased dietary fiber intake were also observed in long term studies of rats [10,11]. Other reports suggest additional benefits to human health in delaying the emergence of some types of colon cancers and in regulating glucose and lipid absorption across the gut [12]. It is also used as a natural laxative.

Diets that are high in insoluble fiber may aid in glycemic control [13,14]. In addition, there are reports that fiber from specific plants, that is, bagasse from sugarcane, may affect carbohydrate and lipid metabolism [15]. Moreover dietary fiber has been reported to decrease the risk of heart disease. Soluble fiber also lowers the cholesterol by binding with bile acids.

A common dietary fiber is psyllium, a natural, water-soluble, gel-reducing fiber extracted from the husks of blond psyllium seeds (Plantago ovata). Psyllium is a member of the class of soluble fibers called mucilages. Mucilages retain water and thus tend to be thick and jelly-like in nature. Psyllium is used as a stabilizing and thickening agent in many soups, salad dressings, lotions and creams. More insoluble fibers, e.g., wheat bran, are classified as cellulose fibers. Additional water-soluble fibers include oat bran and apple pectin which have been shown to lower blood cholesterol.

### Bagasse as Dietary Fiber

Currently bagasse has limited use as a food additive, primarily because of the large amount of crude fiber. The grinding or pulverizing of bagasse for use as dietary fiber has been proposed although the resultant fiber size was hundreds of microns. (U.S. Pat. No. 3,572,593). The process of particle size reduction was to pulverize dry bagasse until all particles passed through a 10-mesh screen size, or about 2000 microns. (For conversion between mesh size and particle size, see the website: http://www.wovenwire.com/reference/particle-size; accessed Feb. 14, 2008.) The particles were then divided into a coarse fraction and a fine fraction. The fine fraction consisted of all particles that would pass through a 40-mesh and an 80-mesh screen size, or about 388 microns and about 177 microns, respectively. The fine fraction was shown to have substantially less crude fiber, but higher protein content than the coarse fraction. The coarse fraction contained the larger portion of nondigestible material.

A method for making a stabilizing agent of highly dispersible cellulose, using bagasse as one example, has been reported. (European Patent Application No. EP 1 839 499 A1) The method started with bagasse straw pulp and was sequentially reduced in size while maintaining fibers with a major to minor axis ratio of about 20 to 300. The smallest major axis reported was 1 to 12 microns. The bagasse was initially cut into smaller pieces and then treated with sodium carboxymethyl cellulose. This mixture was then subjected to nine passes with a high-pressure homogenizer. The final product was a cellulose slurry that was a mixture of bagasse cellulose and sodium carboxymethyl cellulose.

Other methods for producing dietary fiber from plant material have been described. Many of these involve some chemical or heat pretreatment of the plant material prior to particle size reduction. (See, e.g., U.S. Pat. Nos. 5,137,744; 5,403,612; and 4,599,237) For example, a dietary fiber from carrots that retains a high water absorption or binding capacity has been obtained by bleaching the carrot material, drying the material, and then milling. This process produced a fiber that was preferably less than 100 μm. (U.S. Patent Application Publication No. 2003/0044509; and U.S. Pat. No. 6,645,546). In addition, dietary fiber from tapioca pulp fiber has been produced using an enzymatic debranching process. This smallest fibers from this process were retained on a 270-mesh screen, or were larger than about 53 μm. (U.S. Pat. No. 5,350,593). Psyllium particles have been reported as about 15% larger than 80 mesh (about 177 μm), at least about 45% within the range of 80 mesh to about 200 mesh (about 74 μm), and less than about 40% smaller than about 200 mesh. (See U.S. Pat. Nos. 5,149,541 and 5,445,831). A method to make barley flour barley is reported to reduce the barley to sizes wherein about 90% of the flour is less than 50 microns. (U.S. Pat. No. 5,063,078). Because of the difficulties in producing nano-sized particles, an apparatus to grind particles to ultrafine sizes below one micron has been reported which cools the grinding apparatus to temperatures below about −30° C., especially below about −50° C. when grinding/mixing with water ice, and below −80° C. when grinding/mixing with carbon dioxide ice. (U.S. Pat. No. 6,520,837)

There is a need to produce unoxidized nano-sized fibers from a common source, e.g., bagasse, for use as a dietary fiber. Bagasse has other beneficial chemicals that should be preserved in producing a nutritional supplement. In addition, whether such nano-sized dietary fiber retains the beneficial effect of larger fiber products has not been shown.

I have discovered that nano-sized bagasse fibers can be produced that remain unoxidized and retain the bioactivity of bagasse, and are thus useful as a dietary supplement. The particle size reduction was done without heating or chemically altering the bagasse, and resulted in pure bagasse powder in which 70% of the particles had a diameter less than 1 micron. The resulting bagasse particles were shown to be effective as a nutritional supplement in a mouse model. For size reduction, liquid nitrogen was used to initially cool the bagasse to cryogenic temperature, and then the bagasse was mechanically pulverized into small particles. During the pulverization process, chemical oxidation of bagasse was prevented by maintaining a cold temperature, thus preserving the constituents of bagasse. The final size distribution of the bagasse fiber could be lowered by increasing the pulverizing time. The final product was a bagasse nanofiber (a powder) in which none of the natural components of the starting bagasse, including policosanol, have been removed or chemically altered. Using the bagasse nanofibers, the addition of 5 to 10% fiber did not change the texture of a food product. Moreover, the bagasse powder has a natural color and absorbs color evenly so that it could be used as a natural foundation material for cosmetic products.

### BRIEF DESCRIPTION OF THE DRAWINGS

FIG. 1 illustrates the distribution of bagasse particle size after pulverizing for 15 min at temperatures less than about −50° C. The distribution was obtained using a Malvern Instruments Zetasizer nano ZS (Malvern Instrument, Ltd.; Westborough, Mass.) with the particles suspended in distilled water at 25° C.

FIGS. 2A through 2F illustrate the various variables obtained to measure the Zeta potential in a bagasse nanofiber sample that was pulverized for 15 min at temperatures less than about −50° C. The Zeta potential measurements were obtained using a Malvern Instruments Zetasizer nano ZS (Malvern Instrument, Ltd.; Westborough, Mass.) with the particles suspended in distilled water, at a viscosity of 0.8872, at 25° C.

FIG. 3A illustrates the effect of four diets (HFD—only a high-fat diet; CEL—high-fat diet and 10% cellulose fiber; PSY—high-fat diet and 10% psyllium fiber; SCF—high-fat diet and 10% bagasse nanofiber) on the energy intake (kcal/kg) of male mice over a 12 week feeding time. Each point represents the mean±SEM (n=9).

FIG. 3B illustrates the effect of four diets (HFD—a high-fat diet; CEL—high-fat diet and 10% cellulose fiber; PSY—high-fat diet and 10% psyllium fiber; SCF—high-fat diet and 10% bagasse nanofiber) on the body weight (g) of male mice over a 12 week feeding time. Each point represents the mean±SEM (n=9) (“*” means P<0.05; and “**” means P<0.01).

FIG. 4A illustrates the effect of four diets (HFD—a high-fat diet; CEL—high-fat diet and 10% cellulose fiber; PSY—high-fat diet and 10% psyllium fiber; SCF—high-fat diet and 10% bagasse nanofiber) on total fat mass (FM) of male mice over a 12 week feeding time. Each bar represents the mean±SEM (n=9) (“*” means P<0.05; and “**” means P<0.01).

FIG. 4B illustrates the effect of four diets (HFD—a high-fat diet; CEL—high-fat diet and 10% cellulose fiber; PSY—high-fat diet and 10% psyllium fiber; SCF—high-fat diet and 10% bagasse nanofiber) on free fat mass (FFM) of male mice over a 12 week feeding time. Each bar represents the mean±SEM (n=9).

FIG. 5A illustrates the effect of four diets (HFD—a high-fat diet; CEL—high-fat diet and 10% cellulose fiber; PSY—high-fat diet and 10% psyllium fiber; SCF—high-fat diet and 10% bagasse nanofiber) on fasting plasma glucose (mg/dl) of male mice over a 12 week feeding time. Each bar represents the mean±SEM (n=9) (“*” means P<0.05; and “**” means P<0.01).

FIG. 5B illustrates the effect of four diets (HFD—a high-fat diet; CEL—high-fat diet and 10% cellulose fiber; PSY—high-fat diet and 10% psyllium fiber; SCF—high-fat diet and 10% bagasse nanofiber) on fasting plasma insulin (ng/ml) of male mice over a 12 week feeding time. Each bar represents the mean±SEM (n=9) (“*” means P<0.05; and “**” means P<0.01).

FIG. 6A illustrates the effect of four diets (HFD—a high-fat diet; CEL—high-fat diet and 10% cellulose fiber; PSY—high-fat diet and 10% psyllium fiber; SCF—high-fat diet and 10% bagasse nanofiber) over a 12 week feeding time on plasma glucose (mg/dl) of male mice in an intraperitoneal glucose tolerance test. Each bar represents the mean±SEM (n=9) (“**” means P<0.01; and “***” means P<0.001).

FIG. 6B illustrates the effect of four diets (HFD—a high-fat diet; CEL—high-fat diet and 10% cellulose fiber; PSY—high-fat diet and 10% psyllium fiber; SCF—high-fat diet and 10% bagasse nanofiber) over a 12 week feeding time on the area under the curve (AUC) for plasma glucose (mg/dl) of male mice in an intraperitoneal insulin tolerance test. Each bar represents the mean±SEM (n=9) (“*” means P<0.05; “**” means P<0.01; and “***” means P<0.001).

FIG. 7 illustrates the effect of four diets (HFD—a high-fat diet; CEL—high-fat diet and 10% cellulose fiber; PSY—high-fat diet and 10% psyllium fiber; SCF—high-fat diet and 10% bagasse nanofiber) over a 12 week feeding time on stomach ghrelin mRNA expression level (as % of HFD) of male mice measured by real-time reverse transcriptase-polymerase chain reaction and normalized using β-actin. Each bar represents the mean±SEM (n=9) (“*” means P<0.05; and “***” means P<0.001).

FIG. 8 illustrates the effect of four diets (HFD—a high-fat diet; CEL—high-fat diet and 10% cellulose fiber; PSY—high-fat diet and 10% psyllium fiber; SCF—high-fat diet and 10% bagasse nanofiber) over a 12 week feeding time on plasma leptin levels (pg/ml) of male mice. Each bar represents the mean±SEM (n=9) (“*” means P<0.05; and “***” means P<0.001).

FIG. 9 illustrates the effect of four diets (HFD—a high-fat diet; CEL—high-fat diet and 10% cellulose fiber; PSY—high-fat diet and 10% psyllium fiber; SCF—high-fat diet and 10% bagasse nanofiber) over a 12 week feeding time on plasma glucagon-like peptide 1 (GLP-1; pmol/ml) of male mice. Each bar represents the mean±SEM (n=9) (“***” means P<0.001).

A new method to process sugarcane bagasse has been discovered to produce nano-sized fibers (<1 μm) that should retain all the natural components of the starting bagasse. The bagasse is never heated or chemically altered. The bagasse remains cooled to temperatures <−50° C. throughout the size reduction process. The bagasse nanofiber powder was added to a food product, bread, without a problematic change in color or texture. This indicates these fibers would be useful as dietary fiber to add to any food product, including without limitation, ice cream, bread, cookies, noodles, flour, pasta, etc. Dietary fiber of bagasse nanofibers was also shown to retain the bioactivity reported for dietary fiber of larger bagasse fibers (in the micron size) and of other known plant material. The bagasse nanofiber was shown to improve glucose levels, lower insulin and attenuate weight gain in a mouse model of diet-induced obesity. Dietary fiber of bagasse nanofibers can easily be added to other vitamins or minerals to form compositions as dietary supplements, either in solid or liquid form. In addition, bagasse nanofibers make a fine powder that could be used as baby powder or in the cosmetic industry to make foundation or other powder-based products.

### EXAMPLE 1

### Processing the Sugarcane Bagasse and Resulting Fiber Analysis

Bagasse was collected from the field and washed to remove the dirt and soil. The clean bagasse was dried using hot air or other common drying techniques. The dry bagasse and the pulverizing apparatus were both cooled by adding liquid nitrogen, dry ice (CO₂), or other methods to a temperature usually lower than −50° C., preferably lower than −80° C. The preferred temperature is sufficiently low to prevent the oxidation of the bagasse components during the processing. In addition, the low temperature makes the bagasse fibers brittle allowing the fibers to be pulverized into nanometer sized fibers without damaging useful natural chemical compounds, e.g., policosanol. The fibers were ground using a ball milling or a grinding machine. Any pulverizing or grinding machine could be used as long as the temperature can be maintained at preferably lower than −50° C. To maintain the low temperature, liquid nitrogen or dry ice can be applied either inside or outside the pulverizing machine system, with the preferred method applying inside the pulverizing machine system together with the bagasse. In this preferred way, the temperature remained sufficiently cool to prevent the oxidation of components while reducing the size of the bagasse fibers.

The milling time, the amount of impact ball loading, and the grinding speed will determine the particle size of the final product. At a temperature of −80° C., ball milling led to a final particle size that depended on the time of processing as shown in Table 1. The final powder was weighed before and after passage through a 1 μm filter to determine the percentage of particles that were less than 1 μm. In addition, the processing time changed the soluble and insoluble fiber ratio of the resulting powder, i.e., the longer the processing time, the more percent soluble fiber. The following table gives the results:

TABLE 1
Particle Size As a Function of Processing Time

| PROCESSING TIME | PARTICLES <1 μM |
|---|---|
| (MIN) | (%) |
| 15 | 70 |
| 30 | 74 |
| 45 | 78 |
| 60 | 80 |

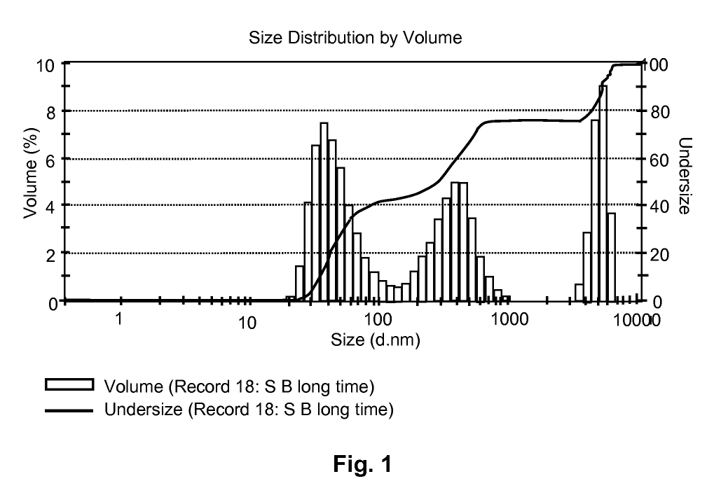

A sample of the powdered bagasse processed by ball milling for 15 min was sent to Malvern Instrument, Ltd. (Westborough, Mass. ) for an analysis of particle size and Zeta potential using a Malvern Instruments Zetasizer nano ZS. The particle size of the bagasse nanofibers was measured in deionized water of pH=7.0 at 25° C. using dynamic light scattering. The results are shown in FIG. 1 and in Table 2. The particles had mainly three size ranges with mean diameter values of 49 nm (44% by volume), 398 nm (32% by volume),and 5220 nm (24% by volume), respectively. This analysis confirms the earlier experiment showing that over 70% of the particles have a size less than 1 μm (or 1000 nm).

TABLE 2
Characteristics of the Three Peaks in FIG. 1.

| | | VOLUME | |
|---|---|---|---|
| | MEAN DIAMETER | OF | WIDTH |
| | OF PEAK | SAMPLE | OF PEAK |
| | (NM) | (%) | (NM) |
| Peak 1 | 49.4 | 44.2 | 22.9 |
| Peak 2 | 398 | 31.8 | 154 |
| Peak 3 | 5220 | 24.0 | 748 |

A Zeta diagnostics was conducted by the same laboratory. As shown below, the particles have at least two mean Zeta potential values of −29.7 mV and −46.7 mV, respectively. Zeta potential, in colloidal chemistry, refers to the electrostatic potential generated by the accumulation of ions at the surface of the colloidal particle which is organized into an electrical double-layer consisting of the Stern layer and the diffuse layer. The zeta potential of a particle can be calculated if the electrophoretic mobility of the sample is known by Henry's Equation:

$${U}_{e} = \frac{2 ɛζ f ( ka )}{3 η}$$

Where Uₑ is the electrophoretic mobility, ε is the dielectric constant of the sample, ζ is the zeta potential, f(ka) is Henry's Function (most often used are the Huckel and Smoluchowski approximations of 1 and 1.5, respectively), and η is the viscosity of the solvent.

The primary relevance of the zeta potential of a colloid is as a relative measure of the stability of the system being measured. The DLVO theory for colloidal interactions dictates that a colloidal system will remain stable if and only if the Coloumbic repulsion arising from the net charge on the surface of the particles in a colloid is greater than the Van der Waals force between those same particles. When the reverse is true, the colloidal particles will cluster together and form flocculates and aggregates (depending on the strength of the Van der Waals attraction and the presence/absence of Steric effects). Since the higher the absolute zeta potential, the stronger the Coloumbic repulsion between the particles, and therefore the lesser the impact of the Van der Waals force on the colloid.

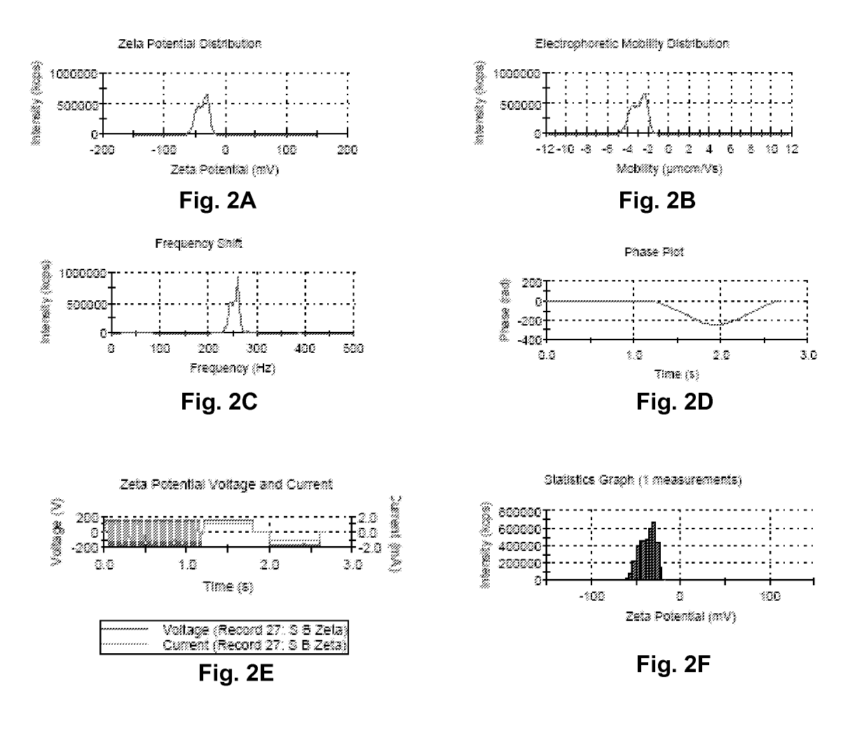

In general, a Zeta potential below −20 mV in water indicates that the colloidal system is relatively stable. For the bagasse sample, the analysis was conducted in water, at a viscosity of 0.8872, and a temperature of 25° C. The number of runs was 22 at a measurement position of 2.00 mm. The results are shown graphically in FIGS. 2A through 2E. A summary is given in Table 3. As shown in FIGS. 2A through 2E and in Table 3, the bagasse nanofibers in water indicated two peaks with zeta potential values of −29.7 mV and −46.7 mV. These values indicate that these nano-sized powders are stable in water and will not aggregate easily.

TABLE 3
Zeta Diagnostics for Bagasse Nanoparticles

| | Mean Zeta | | |
|---|---|---|---|
| | For Peak | Area of Peak | Width of Peak |
| | (mV) | (%) | (mV) |
| Peak 1 | −29.7 | 52.8 | 3.56 |
| Peak 2 | −46.7 | 47.2 | 4.72 |

Values for Sample:

| | |
|---|---|
| Zeta Potential (mV) | −37.7 |
| Zeta SD (mV) | 8.85 |
| Mobility (μmcm/Vs) | −2.95 |
| Mobility SD (μmcm/Vs) | 0.694 |
| Wall Zeta Potential (mV) | −34.5 |
| Effective Voltage (V) | 150 |
| Conductivity (mS/cm) | 0.521 |

The bagasse nanofibers produced as described above are all natural fibers with no changes due to treatment by heating or chemicals. Cooling the bagasse initially to very low temperatures and maintaining low temperatures during processing preserves the components of bagasse, including policosanol, for example. Thus one advantage of the bagasse nanofibers as produced is the retention of any beneficial components in the bagasse. A second advantage is that nano-sized fibers can easily be added into food without much change in texture or taste.

### EXAMPLE 2

### Bagasse Nanofiber as Food Additive

To determine the effects of the bagasse nanofibers on food, yeast bread was made with pure flour, and with flour mixed with three different concentrations of bagasse nanofibers (5%, 7.5%, and 10%). The nanofibers were produced with 15 min processing time. The four flours were then used to make bread using the same process. The color and hardness of the breads was determined by procedures known in the field by Dr. Zhimin Xu, Department of Food Science, Louisiana State University and Agricultural and Mechanical College. The results are shown in Table 4 below:

TABLE 4
Color and Texture of Bread with Bagasse Nanofibers as Additive
COLOR

| | | | | E (Color |
|---|---|---|---|---|
| | L | A | B | difference |
| | (0 = black; | (−a = green; | (−b = blue; | from control |
| Treatment | 100 = white) | +a = red) | +b = yellow) | (all flour)) |
| Control (All | 79.33 ± 1.03 | −0.62 ± 0.32 | 22.84 ± 0.62 | |
| flour) | | | | |
| 5% Bagasse | 69.97 ± 1.63 | 1.54 ± 0.22 | 25.75 ± 0.44 | 10.34 ± 1.08 |
| 0.075 | 67.22 ± 1.01 | 1.98 ± 0.19 | 26.13 ± 0.66 | 12.84 ± 1.18 |
| Bagasse | | | | |
| 10% Bagasse | 65.54 ± 1.06 | 2.49 ± 0.21 | 26.42 ± 0.45 | 14.60 ± 1.10 |

TEXTURE/HARDNESS (Less hardness is softer bread)

| Treatment | Hardness (g) | Hardness (N) |
|---|---|---|
| Control (All | 706.85 ± 217.34 | 6.93 ± 2.13 |
| flour) | | |
| 5% Bagasse | 645.03 ± 111.56 | 6.33 ± 1.09 |
| 7.5% Bagasse | 716.79 ± 115.32 | 7.03 ± 1.13 |
| 10% Bagasse | 818.44 ± 135.11 | 8.03 ± 1.33 |

As shown above, the bread made with the bagasse nanofibers was very similar in color and texture to the control. A small increase in red and yellow color was seen with the increase in percent bagasse nanofibers. However, to the human eye, the color appeared only slightly darker. There was no significant difference in the softness of the bread. It is also believed that the addition of bagasse nanofibers would not affect the taste of food products as much as addition of larger dietary fibers. Thus these bagasse nanofibers are an excellent source of dietary fiber to add to food products. The following experiments show that the bagasse nanofibers retain the beneficial effect of dietary fiber despite the small size.

### EXAMPLE 3

### Study Design and Methods for Mice Fed Various Dietary Fibers

Three kinds of dietary fibers, i.e., sugar cane bagasse nanofiber (SCF), psyllium (PSY), and cellulose (CEL), were compared for the metabolic effects on body weight, plasma insulin, glucose and lipids in a high-fat diet fed mouse model.

### Study Design

Thirty-six male 4-week-old C57BL/6 mice were obtained from the Jackson Laboratory (Bar Harbor, Me.). After arrival, the animals were housed one per cage with ad libitum access to rodent chow and water for a 2-week acclimation period under specific pathogen-free conditions and 12-hour light-dark cycle. Animals were then randomly divided into 4 treatment groups that consisted of high-fat diet alone (HFD), high-fat diet containing 10% (wt/wt) cellulose (CEL), high-fat diet containing 10% (wt/wt) psyllium (PSY), or high-fat diet containing 10% (wt/wt) sugarcane nanofiber (SCF). Each mouse group was fed the assigned diet for 12 weeks. The sources for cellulose, psyllium and sugarcane nanofibers are given below. The high-fat diet was purchased from Research Diets (D-12331, New Brunswick, N.J.) and contained 58% of energy from fat. This high-fat diet has been well documented to induce obesity and insulin resistance in the mice animal model [16]. The components and energy density of these diets are demonstrated in Table 5. Body weight and food intake were measured weekly. In addition, body composition and other measures of carbohydrate metabolism were assessed at the end of study. At the end of the study, overnight fasted mice were euthanized. Plasma, stomach, and other tissues were quickly put into liquid nitrogen container and stored at −80° C. for later analysis. The Institutional Animal Care and Use Committee of Pennington Biomedical Research Center approved all animal protocols.

TABLE 5
Diet Composition And Energy Density Of 4 High-Fat Diets
With Or Without Adding 10% Of Dietary Fibers (mean (SEM))

| CHARACTERISTIC | HFD | 10% CEL | 10% PSY | 10% SCF |
|---|---|---|---|---|
| Casein, 80 mesh | 228 | 205.2 | 205.2 | 205.2 |
| DL-Methionine | 2 | 1.8 | 1.8 | 1.8 |
| Maltodextrin 10 | 170 | 153 | 153 | 153 |
| Sucrose | 175 | 157.5 | 157.5 | 157.5 |
| Soybean oil | 25 | 22.5 | 22.5 | 22.5 |
| Coconut oil, hydrogenated | 333.5 | 300.1 | 300.1 | 300.1 |
| Mineral mix S10001 | 40 | 36 | 36 | 36 |
| Sodium bicarbonate | 10.5 | 9.45 | 9.45 | 9.45 |
| Potassium citrate | 4 | 3.6 | 3.6 | 3.6 |
| Vitamin mix V10001 | 10 | 9 | 9 | 9 |
| Choline bitartrate | 2 | 1.8 | 1.8 | 1.8 |
| Fiber (carbohydrate g) | 0 | 100 | 100 | 100 |
| | | (8.7) | (18.4) | (32.5) |
| Fiber: Insoluble fiber (%) | 0 | 99.5 | 58 | 86 |
| Fiber: Soluble fiber (%) | 0 | 0.5 | 42 | 14 |
| Energy (kcal/kg) | 5558.5 | 5037.5 | 5058.5 | 5132.7 |
| Energy from fat (%) | 58 | 52.2 | 52.2 | 52.2 |

### Source of Dietary Fibers

Psyllium husk powder was obtained from Source Naturals (Scotts Valley, Calif.). Dietary fiber cellulose powder was obtained from NutriCology (Hayward, Calif.). Sugarcane fiber was obtained and pulverized for 15 min as described above. As shown in Table 5, the sugarcane nanofibers had 86% insoluble fiber and 14% soluble fiber, as compared with psyllium powder with 58% insoluble fiber and 42% soluble fiber. Psyllium powder is known to be a soluble fiber and a good laxative.

### Blood Chemistry and Hormone Analysis

After 4 hours of fasting, blood samples were collected from the orbital sinus of unconscious mice induced by inhalation of CO₂. Plasma glucose level was measured by a colorimetric hexokinase glucose assay (Sigma Diagnostics, St Louis, Mo.). Plasma insulin level was determined by an ultrasensitive rat insulin enzyme-linked immunosorbent assay (ELISA) kit from Crystal Chem (Downers Grove, Ill.). Plasma leptin was determined by using Mouse Serum Adipokine LINCOplex Kit (catalog no. MADPK-71K, Linco Research, St Charles, Mo.), and plasma glucagon-like peptide-1 (GLP-1) concentration was measured by GLP-1 (active) ELISA kit (catalog no. EGLP-35k; Linco Research). All assays were conducted in duplicate and according to the manufacturer's instructions.

### Body Composition Measurement

Body composition for all animals was measured by nuclear magnetic resonance [17]. Total fat mass (FM) and free fat mass (FFM) were recorded.

### Assessment of Carbohydrate Metabolism

The effect of the diets on insulin and glucose parameters were determined with the use of an intraperitoneal glucose tolerance test (IPGTT) and insulin tolerance test (IPITT) obtained at week 11 and week 12 of the study, respectively. After an overnight fast, IPGTT was performed by intraperitoneal injection of 2 g glucose (20% glucose in 0.9% NaCl) per kilogram body weight; and blood glucose was measured at the designated times as described [18]. For IPITT, an intraperitoneal injection of human insulin (Eli Lilly, Indianapolis, Ind.) at a dose of 0.75 U/kg body weight was administered after 4 hours of fasting. Whole blood glucose was measured from the tail vein at 0, 30, 60, 90, and 120 min after injections for both IPGTT and IPITT using the FreeStyle blood glucose monitoring system (TheraSense, Phoenix, Ariz.).

### Quantitative Reverse Transcriptase—Polymerase Chain Reaction Procedure

Total RNA was extracted from gastric tissues using TRIzol Reagent (Invitrogen, Carlsbad, Calif.). The RNA analysis and quantitation were performed with RNA 6000 Nano LabChip kit (Agilent Technologies, Foster City, Calif.). Amplification of mouse ghrelin was performed with the Brilliant QRT-PCR 1-step master mix kit (catalog no. 60055; Stratagene, Cedar Creek, Tex.), and cyclophilin B messenger RNA (mRNA) was measured by SYBR Green QPCR master kit (catalog no. 600548, Stratagene) according to the manufacturer's protocol. After each run, a relative quantification of the amplified polymerase chain reaction product in the different samples was measured. A standard curve was used to obtain the relative concentration of the target gene (data not shown), and the results were corrected according to the concentration of cyclophilin B. The results were expressed as percentage of HFD group, setting the mean of the control group at 100% and then calculating each individual value of the other 3 groups of animals studied. TaqMan primer-probe sets of mouse ghrelin (NM_{—}01190296, catalog no. 445046) were purchased from Applied Biosystems (Foster City, Calif.). Primers for mouse cyclophilin B were designed by using PRIMER EXPRESS software (Applied Biosystems). The target gene primer pairs are as follows: for mouse cyclophilin (NM_{—}011149), forward, 5′-TGGAGAGCACCAAGACAGACA-3′ (SEQ ID NO: 1) and reverse, 5′-GTCGACAATGATGACATCCTTCA-3′ (SEQ ID NO: 2). These primers were obtained from Integrated DNA Technologies (Coralville, La.).

### Statistical Analysis

All data are expressed as mean±SEM. Data were evaluated for statistical significance by a 2-way analysis of variance, and P<0.05 was considered significant.

### EXAMPLE 3

### Results of Feeding Dietary Fiber Diets to Mice

### Food Intake, Body Weight, and Body Composition

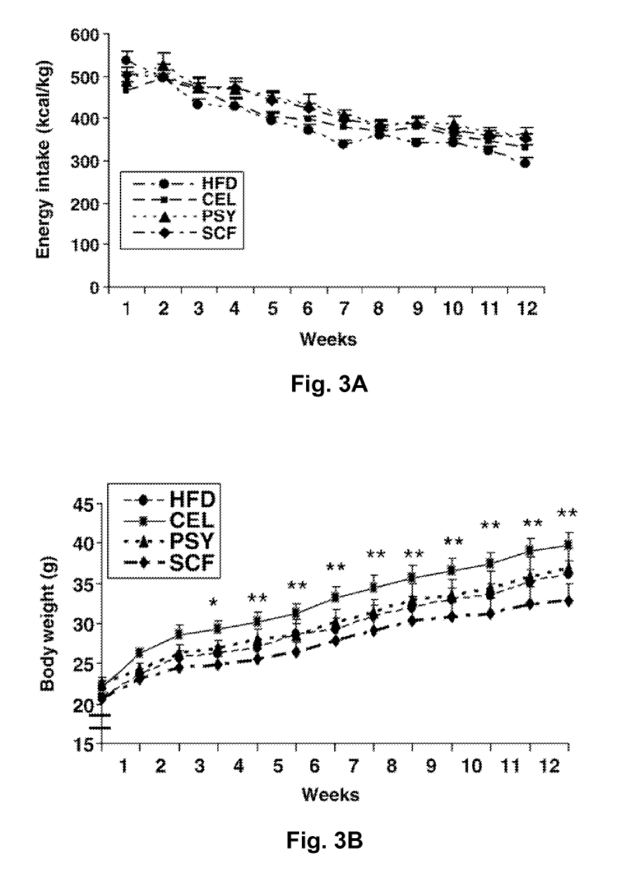

Energy intake and body weight gain in the high-fat diet-fed mice with and without supplementation of dietary fibers are shown in FIGS. 3A and 3B. Energy intake is expressed as kilocalories per kilogram of body weight for 12 weeks. As shown in FIG. 3A, energy intake (in kilocalories per kilogram), normalized by body weight, was not shown to differ among the groups. The average energy intake in all groups expressed per unit body weight was reduced by about 35% at the end of the study when compared with baseline.

In FIG. 3B, the body weight is shown as the mean±SEM, with 9 nine mice in each group. Statistical analyses were done to see if a difference was found between PSY (psyllium) and SCF (sugarcane nanofiber) versus HFD (high-fat diet only) and CEL (cellulose). An * indicates a difference of p<0.05 and an ** indicates a difference of p<0.01. There was no difference in body weight or body composition between the 4 groups at the beginning Beginning at week 3, the body weights of the SCF and the PSY groups were observed to be lower than that of the CEL group (P<0.01 and P<0.05, respectively); and this trend continued up to the end of study (FIG. 3B). At the end of the study, the net body weight gain (mean±SEM) was 12.4±1.03 g for the SCF group, 14.38±0.88 g for HFD alone, 14.4±1.6 g for the PSY group, and 16.7±1.3 g for the CEL groups. The net body weight gains in the SCF, HFD, and PSY groups were significantly less than that in the CEL group (P<0.01, P<0.05, and P<0.05, respectively). Although not a significant difference at 12 weeks, it is believed that if the experiments were run over a longer period of time, the SCF group would show a decrease in net body weight gain from the HFD group.

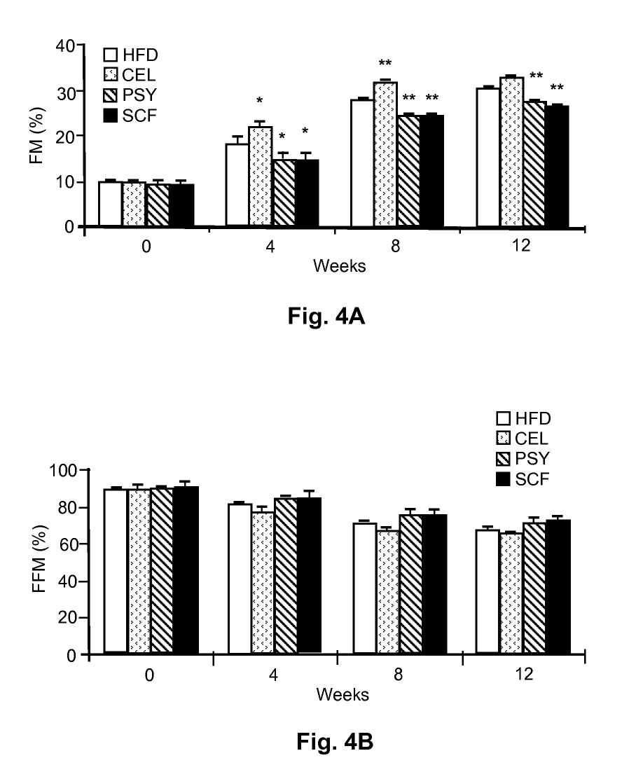

Body composition was measured for total fat mass (FM) and free fat mass (FFM) using nuclear magnetic resonance. The results are shown in FIGS. 4A (FM) and 4B (FFM) as the mean±SEM, for 9 mice in each group. Body composition analysis showed that the FM of the SCF and PSY groups was significantly lower than that of the CEL group (P<0.05, and P<0.05, respectively), but there were no significant differences between the HFD and PSY groups (FIG. 4A). The FFM for all groups was not significantly different (FIG. 4B), except for the CEL group at week 8 (P<0.05), when compared with the HFD group.

### Glucose and Insulin Levels

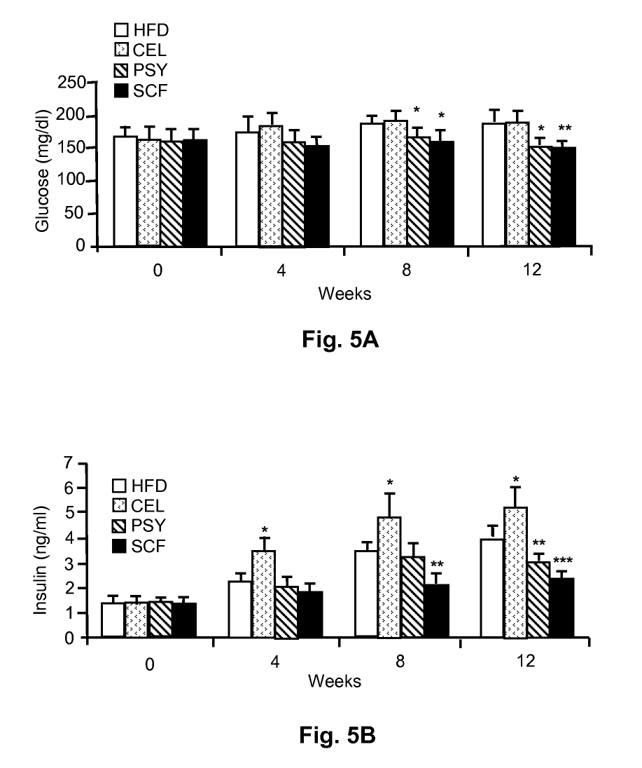

Effect of dietary fiber supplementation on fasting plasma glucose and insulin concentrations in high-fat diet-fed mice for 12 weeks are shown in FIGS. 5A and 5B. Blood samples were collected at week 0 and every 4 weeks after 4 hours of fasting. FIG. 5A shows the plasma glucose levels, and FIG. 5B shows the insulin levels. Data are presented as mean±SEM. Fasting glucose levels were significantly lower in the PSY and SCF groups than those in the CEL and HFD groups beginning at 8 weeks and continuing up to the end of study (FIG. 5A). Fasting plasma insulin was much lower in the PSY and SCF groups than that in the CEL group from week 4 and was maintained to the end of the study (P<0.05 and P<0.01, respectively). However, insulin concentration in the CEL group was significantly higher than that in the HFD group from week 4 to week 12 (P<0.05). Insulin level was substantially lower in the SCF group than in the HFD group, and there was no difference between the PSY and HFD groups (FIG. 5B).

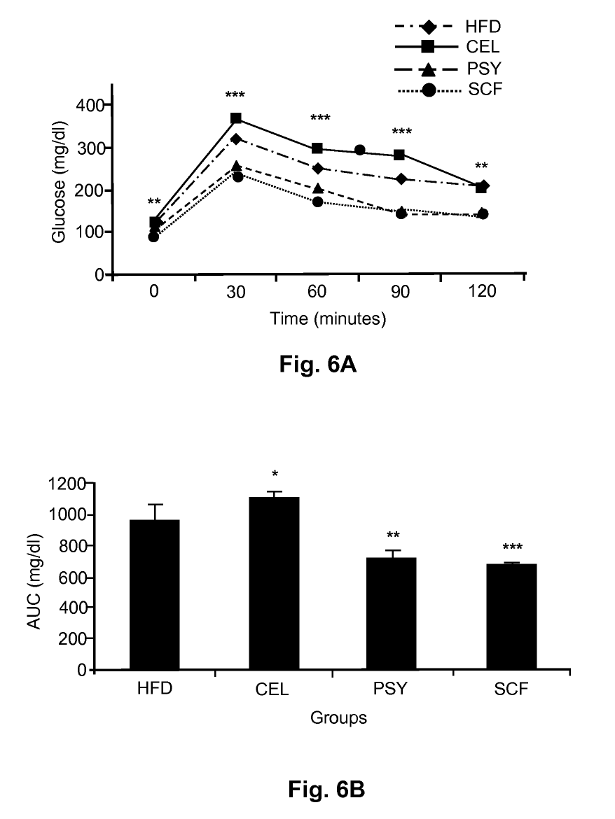

The effects of dietary fiber and high-fat diet on an intraperitoneal glucose tolerance test (IPGTT) and an insulin tolerance test (IPITT) are shown in FIGS. 6A and 6B. The IPGTT was done after overnight fasting as described above. As shown in FIG. 6A, the IPGTT data showed glucose concentrations were much lower in the PSY and SCF groups than in the control and CEL groups (P<0.01 and P<0.001, respectively). In FIG. 6B, the IPITT was performed after 4 hours of fasting (AUC=area under curve of IPITT). The area under the curve for glucose during the IPGTT was 945±115 mg/dL in HFD, 110±36 mg/dL in CEL, 724±39 mg/dL in PSY, and 667±24 mg/dL in SCF. The IPITT results in these groups was lower in the PSY and SCF groups than in the control and CEL groups (P<0.01 and P<0.001, respectively) (FIG. 6B).

### Stomach Ghrelin Gene Expression Analysis

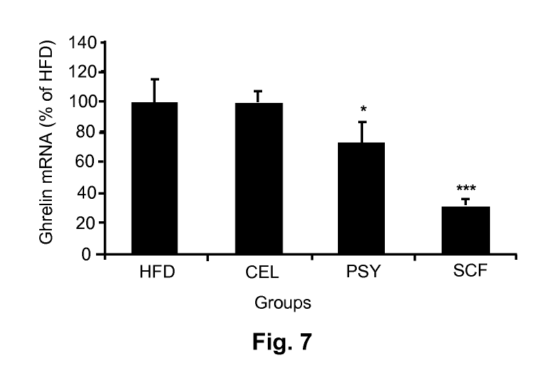

Ghrelin is an endogenous ligand for the growth hormone secretagogue receptor (GHSR). Accumulating evidence has suggested that ghrelin may play a role in signaling and reversing states of energy insufficiency. Ghrelin levels rise after food deprivation, and ghrelin administration stimulates feeding and increases body weight and adiposity [21,22]. Stomach ghrelin gene expression in the high-fat diet-fed mice with and without dietary fiber supplementation was measured by real-time reverse transcriptase-polymerase chain reaction in triplicate, and the results were normalized using β-actin. The results are shown in FIG. 7 as a mean±SEM. The stomach ghrelin mRNA levels were not statistically different between the HFD and CEL groups. However, the ghrelin gene expression levels in the PSY and the SCF animals were significantly lower than that in the HFD and CEL animals (P<0.05 and P<0.001, respectively) as shown in FIG. 7.

### Effect of Dietary Fiber Supplementation on Plasma Leptin Concentration

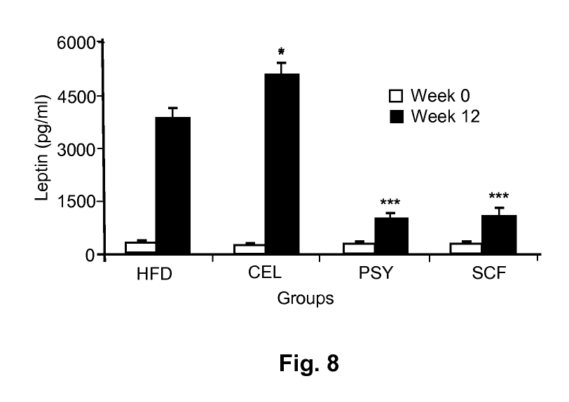

The effect of dietary fiber in a high-fat diet on plasma leptin levels were measured. Fasting plasma leptin was measured at week 0, and then at week 12 in all four mice groups. The data are shown in FIG. 8 as a mean±SEM. At baseline (week 0), there was no difference in plasma leptin level among all groups. At week 12, leptin levels increased from basal 358±38 to 3871±279 pg/mL in the HFD, from 286±64 to 5054±370 pg/mL in the CEL, from 309±42 to 1020±196 pg/mL in the PSY, and from 319±62 to 1097±256 pg/mL in the SCF. Plasma leptin concentrations were significantly lower in the SCF and PSY groups than in the CEL and HFD groups (mean±SEM, P<0.001; FIG. 8). Leptin level at week 12 in the CEL group was significantly higher than that in the HFD group (P<0.05).

### High-Fat Diet and Dietary Fiber Affect Plasma GLP-1 Level

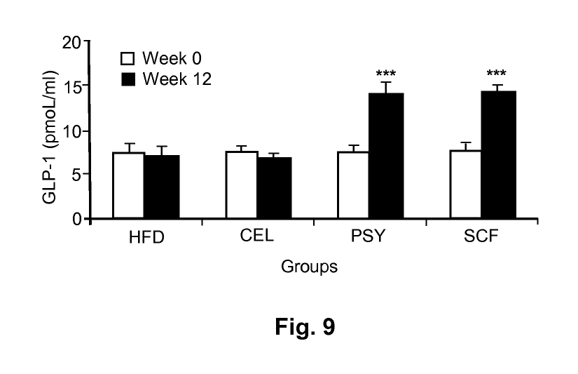

Glucagon-like peptide 1 is secreted from enteroendocrine L cells, which are localized in the distal ileum and colon [23]. Glucagon-like peptide 1 acts through a specific G-protein-coupled receptor to potently stimulate glucose-dependent insulin secretion [24]. Glucagon-like peptide 1 further reduces hyperglycemia through inhibition of both glucagon secretion and gastric emptying [25-27]. The effect of dietary fiber on fasting plasma glucagon-like peptide (GLP-1) concentration was determined. Plasma GLP-1 was determined by GLP-1 ELISA kit (as described above) at week 0 and at week 12. The data are shown in FIG. 9 as mean±SEM. There was no difference in fasting plasma GLP-1 concentrations among the four groups at week 0. After 12 weeks of feeding, GLP-1 levels slightly decreased in the HFD and CEL groups (−4.5% and −8.9%, respectively) and significantly increased in the PSY and SCF groups (+85% and +87.7%, respectively; P<0.001) over the baseline levels. (FIG. 9).

The above data indicate that high-fat diets containing a larger percentage of soluble fiber, such as provided in the diet with sugarcane fiber or psyllium, resulted in lower glucose and insulin levels in this animal model. Specifically, fasting plasma glucose and insulin levels during the study were observed to be significantly lower in the SCF and PSY groups than in the CEL groups. The mechanism is not precisely known, but a contributing factor may be the altering of the rate of glucose absorption in the gut. Dietary fiber, particularly soluble fiber found in barley and oats, may slow digestion and absorption of carbohydrates and hence lower blood glucose and insulin levels. The body composition analysis also revealed that diets incorporating either SCF or PSY fiber, as opposed to cellulose, appeared to attenuate weight gain from ingestion of a high-fat diet. The effectiveness of the bagasse nanofiber of lower fasting glucose in this study was similar to results in streptozotocin-induced diabetic rats fed a diet containing 5% fiber (not using nanofibers). The plasma glucagon levels were decreased in bagasse and significantly increased in the control animals, whereas plasma insulin levels were not changed in these groups [20]. However, in that study, body weight gain was greater for the sugarcane fiber as opposed to the results observed in this study. These results indicate that the bagasse nanofibers are beneficial as dietary fiber, and may have greater benefits on body weight than the previously used dietary fiber made of bagasse.

In addition to the weight and carbohydrate parameters, several biochemical parameters, such as ghrelin and GLP-1 levels, were altered in the diets containing primarily sugarcane fiber or psyllium. The above data suggested that high-fat diets containing 10% of either the sugarcane fiber or psyllium significantly lowered stomach ghrelin mRNA levels when compared with high-fat diets alone or high-fat diets containing 10% cellulose. In addition, high-fat diets containing primarily sugarcane and psyllium resulted in lower plasma leptin concentrations when compared with high-fat diet alone or high-fat diet containing 10% cellulose. GLP-1 has a regulatory effect on energy intake by decreasing food intake and promoting satiety. [23-27].

In summary, the above data demonstrate that mice fed a high-fat diet containing dietary fibers in the form of bagasse nanofibers was effective in improving glucose levels, lowering insulin, and attenuating weight gain in a model of dietary-induced obesity when compared with high-fat diet alone or high-fat diet supplemented with cellulose. The data indicated that the bagasse nanofiber was as effective as the psyllium dietary fiber, a plant fiber commonly used for fiber.

### EXAMPLE 5

In another experiment, the effects of three dietary fibers—sugarcane bagasse nanofibers (SCF; size<1 um), psyllium (PSY) and cellulose (CEL)—on various energy characteristics were compared. Twenty-seven male mice (2-month old; C57BL/6) were randomly divided into three groups and fed high-fat diets supplemented with 10% SCF, PSY or CEL as described above. The rest of the study design was as described above. At the end of the study, the mice were killed by cervical dislocation and decapitated. Truncal blood was collected and plasma was frozen for further measurements. Also, production of fecal pellets was monitored, and the collected for analysis.

For the blood chemistry, plasma cholesterol and triglycerides were measured by the cholesterol quantitation kit (BioVision, Inc.; Mountain View, Calif.) and a triglyceride kit (Sigma Chemical Co., St. Louis, Mo.). High density lipoprotein cholesterol (HDL-ch) values were measured by a sodium phosphotunstate-MgCl₂ precipitation. The effect of the diets on insulin, glucose, and leptin was determined as shown above. Data were statistically analyzed as described above. Data are presented as mean±SEM (n=9).

Body weight, insulin, glucose and lipids were not statistically different between the groups at week 0 (the baseline) (data not shown). After 8 weeks, body weight gains were 10.1±2.3 g for SCF, 10.9±3.5 for PSY, and 14.4±3.2 for CEL. The body weight gains for the mice eating high-fat diets supplemented with either SCF or PSY were significantly lower than the CEL group (P<0.05 and P<0.01, respectively). Fasting plasma insulin level increased by 1.58-fold in the SCF, 2.44-fold in the PSY, and 3.13-fold in the CEL group compared with their baseline levels, with significantly lower concentration in the SCF than in the CEL group (P<0.05). A similar trend was found in the changes of fasting glucose levels between the groups. (Data not shown).

As shown in Table 6, the frequency of stool production was highest with PSY, a water soluble fiber and known laxative. The lowest stool production was with the SCF diet. Both the glycerol and cholesterol concentration in the feces was increased in the PSY and SCF diets above the CEL (P<0.01 and P<0.05, respectively).

TABLE 6
Effect of Dietary Fibers on Fecal Composition

| FECAL COMPOSITION | CEL | PSY | SCF |
|---|---|---|---|
| Dry fecal weight (mg/day) | 399.8 ± 19.6 | 446 ± 47 | 295 ± 17 |
| Stool frequency (number/day) | 33 ± 3 | 47 ± 4 | 24 ± 2 |
| Fecal glycerol (μM/day) | 463 ± 28 | 657 ± 80 | 515 ± 67 |
| Fecal cholesterol (μM/day) | 11.8 ± 1.3 | 16.3 ± 2.5 | 14.7 ± 2.1 |

As shown in Table 7, cholesterol concentration was significantly decreased in the PSY group compared with the CEL group (P<0.05), but no difference was observed between SCF and CEL groups. Triglyceride levels, LDL-c, and HDL-c were all significantly lower in the PSY and the SCF groups than in the CEL group (P<0.01).

TABLE 7
Effects of Dietary Fiber on Plasma Lipid Profile

| | | | |
|---|---|---|---|
| GROUP | CEL | PSY | SCF |

Week 0

| | | | |
|---|---|---|---|
| Cholesterol | 3.89 ± 0.15 | 4.02 ± 0.15 | 4.02 ± 0.21 |
| (mmol/L) | | | |
| Triglyceride | 62.15 ± 13.56 | 62.15 ± 7.91 | 54.24 ± 9.04 |
| (mmol/L) | | | |
| LDL-c (mmol/L) | 0.88 ± 0.10 | 1.01 ± 0.13 | 1.03 ± 0.18 |
| HDL-c (mmol/L) | 2.62 ± 0.16 | 2.75 ± 0.16 | 2.75 ± 0.16 |

Week 12

| | | | |
|---|---|---|---|
| Cholesterol | 5.74 ± 0.29 | 5.19 ± 0.34 | 5.55 ± 0.26 |
| (mmol/L) | | | |
| Triglyceride | 140.12 ± 26.0 | 118.61 ± 12.63 | 126.5 ± 24.86 |
| (mmol/L) | | | |
| LDL-c (mmol/L) | 3.01 ± 0.36 | 1.97 ± 0.23 | 1.84 ± 0.39 |
| HDL-c (mmol/L) | 2.08 ± 0.21 | 2.67 ± 0.41 | 3.14 ± 0.44 |

These results are similar to those reported above when comparing mice given the dietary fiber supplemented high-fat diet to mice given only the high-fat diet. These results also confirm the laxative properties of PSY, but show that 10% SCF does not have this effect. Again, the above date indicate that the bagasse nanofibers retain bioactivity.

In the claims and specification, “bagasse nanofibers” are fibers in which more than 70% by mass will pass through a filter or a screen with a one micron size opening. As shown in Example 1 above, this method was confirmed by measuring size distribution using dynamic light scattering.

### REFRENCES

[1] Roberts S B, McCrory M A, Saltzman E. The influence of dietary composition on energy intake and body weight. J Am Coll Nutr 2002; 21:140S-5S.
[2] Howarth N C, Saltzman E, Robers S B. Dietary fiber and weight regulation. Nutr Rev 2001; 59:163-9.
[3] Marlett J A, McBurney M I, Slavin J L. Position of the American Dietetic Association: health implications of dietary fiber. J Am Diet Assoc 2002; 102:993-1000.
[4] Albertson A M, Tobelmann R C. Consumption of grain and whole-grain foods by an American population during the years 1990 to 1992. J Am Diet Assoc 1995; 95:703-4.
[5] Alfieri M A, Pomerleau J, Grace D M, Anderson L. Fiber intake of normal weight, moderately obese and severely obese subjects. Obes Res 1995; 3:541-7.
[6] Van Itallie T B. Dietary fiber and obesity. Am J Clin Nutr 1978; 31 (Suppl):543-52.
[7] Burkitt D P, Trowell H C, editors. Refined Carbohydrate Food and Disease. Some Implications of Dietary Fibre. New York, N.Y.: Academic Press; 1975.
[8] Weickert M O, MoHlig M, Schofl C, Otto B, Viehoff H, Koebnick C, et al. Cereal fiber improves whole body insulin sensitivity in overweight and obese women. Diabetes Care 2006; 29:775-80.
[9] Chandalia M, Garg A, Lutjohann D, von Bergmann K, Grundy S M, Brinkley L J. Beneficial effects of high dietary fiber intake in patients with type 2 diabetes mellitus. N Engl J Med 2000; 342:1440-1.
[10] Li J, Kaneko T, Qin L Q,Wang J, Wang Y, Sato A. Long-term effects of high dietary fiber intake on glucose tolerance and lipid metabolism in GK rats: comparison among barley, rice, and cornstarch. Metabolism 2003; 52:1206-10.
[11] Hozumi T, Yoshida M, Ishida Y, Mimoto H, Sawa J, Doi K, et al. Longterm effects of dietary fiber supplementation on serum glucose and lipoprotein levels in diabetic rats fed a high cholesterol diet. Endocr J 1995; 42:187-92.
[12] Schneeman B O, Tietyen J. Dietary fiber. In: Shills M E, Olson J A, Shiks M, editors. Chapter 4. Modern Nutrition in Health and Disease. 8th ed. Philadelphia (Pa): Lea and Febiger; 1994. p. 89-100.
[13] Kimmel S E, Michel K E, Hess R S, Ward C R. Effects of insoluble and soluble dietary fiber on glycemic control in dogs with naturally occurring insulin-dependent diabetes mellitus. J Am Vet Med Assoc 2000; 216:1076-81.
[14] Mahapatra S C, Bijlani R L, Nayar U. Effect of cellulose and ispaghula husk on fasting blood glucose of developing rats. Indian J Physiol Pharmacol 1988; 32:209-11.
[15] Morgan B, Heald M, Atkin S D, Green J, Chain E B. Dietary fiber and sterol-metabolism in rat. Br J Nutr 1974; 32:447-55.
[16] Collins S. Genetic variation to diet-induced obesity in the C57BL/6J mouse: physiological and molecular characteristics. Physiol Behav 2004; 81:243-8.
[17] Tinsley F C, Taicher G Z, Heiman M L. Evaluation of a quantitative magnetic resonance method for mouse whole body composition analysis. Obes Res 2004; 12:150-60.
[18] Freeman H C, Hugill A, Dear N T, Ashcroft F M, Cox R D. Deletion of nicotinamide nucleotide transhydrogenase: a new quantitive trait locus accounting for glucose intolerance in C57BL/6J mice. Diabetes 2006; 55:2153-6.
[19] Jenkins D J, Wolever T M, Leeds A R, Gassull M A, Haisman P, Dilawari J, et al. Dietary fibres, fibre analogues, and glucose tolerance: importance of viscosity. Br Med J 1978; 1:1392-4.
[20] Yamashita S, Yamashita K, Yasuda H, Ogata E. High-fiber diet in the control of diabetes in rats. Endocrinol Jpn 1980; 27:169-73.
[21] Kojima M, Hosoda H, Date Y, Nakazato M, Matsuo H, Kangawa K. Ghrelin is a growth-hormone-releasing acylated peptide from stomach. Nature 1999; 402:656-60.
[22] Yoshihara F, Kojima M, Hosoda H, Nakazato M, Kangawa K. Ghrelin: a novel peptide for growth hormone release and feeding regulation. Curr Opin Clin Nutr Metab Care 2002; 5:391-5.
[23] Drucker D J. Glucagon-like peptide (review). Diabetes 1998; 47:159-69.
[24] Weir G C, Mojsov S, Hendrick G K, Habener J F. Glucagonlike peptide 1 (7-36) actions on endocrine pancreas. Diabetes 1989; 38:338-42.
[25] Ritzel R, Orskov C, Holst J J, Nauck M A. Pharmacokinetic, insulinotropic, and glucagonostatic properties of GLP-1 [7-36 amide] after subcutaneous injection in healthy volunteers: dose-response relationships. Diabetologia 1995; 38:720-5.
[26] Schirra J, Katschinski M, Weidmann C, Schafer T, Wank U, Arnold R, et al. Gastric emptying and release of incretin hormones after glucose ingestion in humans. J Clin Invest 1996; 97:92-103.
[27] Stoffer D A, Kieffer T J, Hussain D J, Drucker M A, Bonner-Weir S, Habener J F, et al. Insulinotropic glucagon-like peptide I agonists stimulate expression of homeodomain protein IDX-1 and increase islet size in mouse pancreas. Diabetes 2000; 49:741-8.

The complete disclosures of all references cited in this specification are hereby incorporated by reference. Also incorporated by reference is the following references which are not prior art: (1) Z.Q. Wang et al., “Effects of dietary fibers on weight gain, carbohydrate metabolism, and gastric ghrelin gene expression in mice fed a high-fat diet,” Metabolism Clinical and Experimental, vol. 56, pp. 1635-1642 (2007); and (2) Z.Q. Wang et al., “Effect of sugar cane fiber (bagasse) on body weight, carbohydrate and lipid metabolism in high fat diet fed mice,” presented at the 10^{th} International Congress of Obesity, Sydney, Australia, Sep. 3-6, 2006. In the event of an otherwise irreconcilable conflict, however, the present specification shall control.

What is claimed:

1. A method for preparing nano-sized bagasse fibers from native sugarcane bagasse, said method comprising:
(a) washing and drying the native sugarcane bagasse;
(b) cooling the dried sugarcane bagasse to a temperature less than about −50° C.;
(c) pulverizing the sugarcane bagasse at a temperature less than about −50° C. for a time sufficient to reduce at least about 70% of the sugarcane bagasse fibers by mass to a size less than about 1 micron;
wherein the resulting nano-sized bagasse fibers are not chemically altered as compared to those in the native sugarcane bagasse.

2. The method of claim 1, wherein the pulverizing time is between about 15 min and about 60 min.

3. The method of claim 1, wherein the pulverizing time is about 15 min.

4. The method of claim 1, wherein step (b), step (c), or both are carried out in the presence of liquid nitrogen.

5. The method of claim 1, wherein step (b), step (c), or both are carried out in the presence of dry ice.

6. The method of claim 1, wherein said pulverizing step comprises ball-milling.

7. The method of claim 1, additionally comprising the step of incorporating the nano-sized bagasse fibers into a food product.

8. The method of claim 7, wherein the food product is selected from the group consisting of soup, salad dressing, ice cream, bread, other baked goods, cereal, pasta, noodles, and flour.

9. The method of claim 7, wherein the mass of said nano-sized bagasse fibers is at least 10% of the mass of the food product.

10. The method of claim 7, additionally comprising the step of incorporating psyllium fiber into the food product.

11. The method of claim 1, additionally comprising the step of incorporating the nano-sized bagasse fibers into a nutritional supplement comprising vitamins or minerals.

12. The method of claim 11, additionally comprising the step of incorporating psyllium fiber into the nutritional supplement.

13. The method of claim 1, additionally comprising the step of incorporating the nano-sized bagasse fibers into a cosmetic product.

14. The method of claim 1, additionally comprising the step of incorporating the nano-sized bagasse fibers into a pigment suitable for use in a cosmetic product.

15. The method of claim 1, additionally comprising the step of feeding a mammal a diet containing the nano-sized bagasse fibers, wherein the fibers are from about 5% to about 15% of the diet by mass; and wherein at least one of the following outcomes then occurs: the fasting plasma glucose of the mammal is lowered, or the mammal's body mass decreases, or the mammal's appetite decreases, or expression of the ghrelin gene in the mammal's stomach decreases, or the plasma leptin level of the mammal decreases, or the plasma glucagon-like peptide-1 level of the mammal increases.

16. The method of claim 15, wherein the nano-sized bagasse fibers are about 10% of the diet by mass.

17. The method of claim 15, wherein the mammal consumes the diet for a time sufficient to lower the mammal's fasting plasma insulin level by at least 10%.

18. The method of claim 15, wherein the mammal consumes the diet for a time sufficient to lower the mammal's body mass by at least 5%.

19. The method of claim 15, wherein the mammal consumes the diet for a time sufficient to reduce stomach ghrelin gene expression by at least 25%.

20. The method of claim 15, wherein the mammal consumes the diet for a time sufficient to reduce the plasma leptin level by at least 25%.

21. The method of claim 15, wherein the mammal consumes the diet for a time sufficient to increase the plasma glucagon-like peptide-1 level by at least 25%.

22. A method comprising feeding a mammal a diet containing nano-sized bagasse fibers, wherein the fibers are from about 5% to about 15% of the diet by mass; wherein the fibers are sugarcane bagasse fibers that are not chemically altered as compared to native sugarcane bagasse, wherein at least about 70% of the fibers by mass have a size less than about 1 micron; and wherein at least one of the following outcomes then occurs: the fasting plasma glucose of the mammal is lowered, or the mammal's body mass decreases, or the mammal's appetite decreases, or expression of the ghrelin gene in the mammal's stomach decreases, or the plasma leptin level of the mammal decreases, or the plasma glucagon-like peptide-1 level of the mammal increases.

23. The method of claim 22, wherein the nano-sized bagasse fibers are about 10% of the diet by mass.

24. The method of claim 22, wherein the mammal consumes the diet for a time sufficient to lower the mammal's fasting plasma insulin level by at least 10%.

25. The method of claim 22, wherein the mammal consumes the diet for a time sufficient to lower the mammal's body mass by at least 5%.

26. The method of claim 22, wherein the mammal consumes the diet for a time sufficient to reduce stomach ghrelin gene expression by at least 25%.

27. The method of claim 22, wherein the mammal consumes the diet for a time sufficient to reduce the plasma leptin level by at least 25%.

28. The method of claim 22, wherein the mammal consumes the diet for a time sufficient to increase the plasma glucagon-like peptide-1 level by at least 25%.
